(19) United States
(12) Patent Application Publication
(10) Pub. No.: US 2010/0292287 A1
(43) Pub. Date: Nov. 18, 2010

(54) Periodontitis treatment

(76) Inventor: Peter F. Kador, Omaha, NE (US)

Correspondence Address: LITMAN LAW OFFICES, LTD., PATENT LAW BUILDING, 8955 CENTER STREET, MANASSAS, VA, 20110, US

(21) Appl. No.: 12/453,557

(22) Filed: May 14, 2009

(51) Int. Cl. A61K 31/4184; A61K 31/352

(52) U.S. Cl. 514/387; 514/456

(57) ABSTRACT

The periodontitis treatment is a method for the prevention and treatment of periodontitis in mammals, including humans and dogs. The mammal may be diabetic or non-diabetic. the method includes the step of administering an effective amount of an aldose reductase inhibitor (ARI). The ARI may be (i) a phenolic derivative, such as quercetin, quercitrin (a 3-oxy-glucose analog of quercetin), rutin, and other polyphenols or bioflavonoids exhibiting an ARI effect; (ii) an acetic acid derivative, such as tolrestat, ponalrestat, etc.; (iii) a cyclic imide (or hydantoin), such as sorbinil, 2-methyl sorbinil, imirestat, etc.; and (iv) one of the phenylsulfonyinitromethtane derivatives, such as ZD-5522. In particular embodiments, the method may include, e.g., administering a diet containing about 0.08% quercetin (about 80 mg/kg/day), about 0.0125% imirestat (about 12 mg/kg/day), or about 0.015% tolrestat (about 20 mg/kg/day).

### BACKGROUND OF THE INVENTION

1. Field of the Invention

The present invention relates to preventing and treating periodontal disease, and particularly to a method for preventing and treating periodontitis in mammals, particularly in dogs.

2. Description of the Related Art In uncontrolled diabetes mellitus (DM) oral complications include the presence of infections, poor healing, gingivitis, and periodontitis. Periodontitis is a lesser known but frequent complication of diabetes mellitus (DM). It is the major cause of tooth loss. Periodontitis is associated with inflammatory or metabolic disorders of tissues surrounding and supporting teeth. It is caused by pathogenic microflora in the biofilm or dental plaque that forms on the teeth on a daily basis. In periodontitis, the inflammation extends deep into the tissues and causes loss of supporting connective tissue and alveolar bone. This results in the formation of soft tissue pockets or deepened crevices between the gingiva and the tooth root. In severe forms tooth loosening and eventually loss of mastication function can occur. The presence of periodontitis can aggravate glycemic control by increasing insulin resistance and by contributing to a worsening of the diabetic state. Its presence in diabetics is also considered to be an independent predictor of ischemic heart disease, death from myocardial infarction, and nephropathy.

The mechanism(s) initiating diabetic periodontitis have not been established, and currently there is no direct treatment for diabetic periodontitis. Dental therapy for diabetics focuses primarily on the control of oral infections. However, diabetics have impaired wound healing, increased monocyte response to dental plaque, and impaired polymorphonuclear leukocyte (PMN) responses. PMNs are found in the central region of the junctional epithelium, which is located at the interface between the gingival sulcus (which is populated with bacteria) and the periodontal soft and mineralized connective tissues that need protection from becoming exposed to bacteria and their products. The function of PMNs is to maintain gingival and periodontal health, but in DM this function is impaired by altered chemotaxis, adherence and phagocytosis. This impairment in the function of PMNs can lead to impaired host resistance to infection.

Similar changes in PMN function have been observed in diabetic rats. The chemotactic response of crevicular PMNs to casein atraumatically applied to the gingival margin is reduced by the chemical induction of diabetes, and insulin administration reverses this decrease. In infection-free Long Evan diabetic rats (type 1), the phagocytotic ability of the PMNs is significantly less as compared to similar non-diabetic rats. This decrease in phagocytic activity is inversely proportional to plasma glucose levels, indicating that hyperglycemia is linked to the impairment of PMN function that results in infections.

With DM, monocytes and macrophages secrete increased levels of the cytokines tumor necrosis factor (TNF)-α, interleukin (IL)-1 and inflammatory PGE₂. The proinflammatory cytokine TNF-α also fosters insulin resistance, especially in obese patients, where it is produced by adipocytes. Obesity is also a significant predictor of periodontal disease. It has been proposed that the inflammatory process in periodontitis that results in increased TNF-α levels also fosters insulin resistance. TNF-α may contribute to insulin resistance by interfering with tyrosine phosphorylation of insulin receptor substrate molecules, an essential step in the signal transduction pathway for insulin. This action impairs the messenger RNA (mRNA) transcription process needed for synthesis of the insulin-responsive glucose transporter protein (GLUT-4) receptor. TNF-α also causes adipocytes to release free fatty acids that can impair insulin signaling. Soluble TNF-α receptors have also been recently observed in non-obese patients with type 2 DM.

Cytokine levels in diabetics also increase in response to oral pathogens. In patients with type 1 DM, the gingival crevicular fluid contains higher levels of PGE₂ and IL-1β, and there are significantly higher levels of TNF-α, PGE₂ and IL-1β in monocytes from these patients. Experimental studies also show that cytokine expression and inflammatory filtrate are stimulated in both type 1 and 2 diabetic mice inoculated with Porphyromonas gingivalis, a common periodontitis bacteria, compared to similarly inoculated control mice. In these mice, no difference in bacterial killing between the diabetic and control groups was observed, suggesting that diabetes may alter bacteria-host interactions by prolonging the inflammatory response. The importance of TNF-α in this process was demonstrated by reversal of the prolonged cytokine expression by the specific TNF-α inhibitor Enbrel. This indicates that cytokine dysregulation associated with prolonged TNF-α expression represents a mechanism through which bacteria may induce a more damaging inflammatory response in diabetic individuals.

Periodontitis is the most common cause of inflammatory bone loss, and the presence of diabetes increases this loss even further. Inoculation of db/db mice with the common periodontitis bacteria Porphyromonas gingivalis resulted in both a reduction of osteoclastogenesis and bone resorption, as well as a reduction of reparative bone formation. These observations suggest that the net loss of bone in DM is caused by a greater suppression of bone formation than bone increase in resorption. The uncoupling of bone formation and resorption appears linked to prolonged apoptosis of bone lining cells, which diminishes the capacity to form new bone. The importance of this diabetes-induced apoptosis has been demonstrated by treating diabetic mice with a pancaspase inhibitor, z-VAD-fmk (N-benzyloxycarbonyl-Val-Ala-Asp-fluoromethyl-ketone), which inhibits apoptosis. With this inhibitor, significant improvements in several healing parameters, including fibroblast density, enhanced mRNA levels of collagen I and III, and increased matrix formation were observed, along with an increase in the number of bone-lining cells and new bone formation.

Prolonged hyperglycemia, the major risk factor in the development of diabetic complications associated with neuropathy, nephropathy and micro- and macroangiopathy, has also been linked to periodontal disease. Although biochemical and pathophysiological observations of diabetic periodontitis include the presence of inflammatory reactions, neutrophil-linked generation of reactive oxygen species (ROS), cytokine activation, apoptosis, leukocyte dysfunction, altered bone formation and resorption, and the presence of advanced glycation end products (AGEs) and their interaction with their specific receptors (RAGE), dental treatment for diabetic periodontal disease is primarily focused on the control of oral infections. Overlooked have been the potential contribution of the enzyme aldose reductase (AR) and the sorbitol pathway, which plays a critical role in the development of diabetic complications associated with neuropathy, nephropathy and micro- and macroangiopathy.

Investigations have shown that many of the complications of diabetes result, at least in part, to abnormalities in glucose metabolism through the polyol pathway.

Normally the bulk of intracellular glucose is metabolized to provide energy by phosphorylation of glucose, catalyzed by hexokinase, to form glucose-6-phosphate, which is further metabolized to useful energy by entry into the Emden-Myerhof pathway, or anaerobic glycolysis. In the diabetic patient, however, insufficient hexokinase is available to metabolize all of the intracellular glucose.

In many tissues of the body, including lens tissue in the eye, an alternative path is available to metabolize glucose. The enzyme aldose reductase (AR) catalyzes the reduction of glucose to sorbitol with hydrogen supplied by NADPH. Sorbitol is then oxidized to fructose by sorbitol dehydrogenase, the hydrogen being accepted by NAD+. However, in the hyperglycemic patient, although sufficient aldose reductase is available to reduce glucose to sorbitol, there is not sufficient sorbitol dehydrogenase to oxidize the sorbitol to fructose.

This leads to an accumulation of sorbitol in the tissues. Sorbitol does not readily diffuse through the tissues and cellular membranes due to its polarity. It is hypothesized that the accumulation of sorbitol produces a hyperosmotic condition, with resulting fluid accumulation in the cells, altering membrane permeability with the development of the pathological conditions noted above. Consequently, considerable attention has focused on the development of aldose reductase inhibitors (ARIs).

A variety of ARIs have been developed. According to one scheme of classification mentioned by de la Fuente and Manzanaro, ARIs include phenolic derivatives, such as quercetin, which has the structure shown in I;

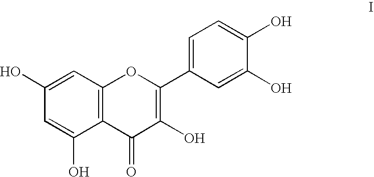

acetic acid derivatives (or more generally, carboxylic acid derivatives), such as tolrestat (structure II);

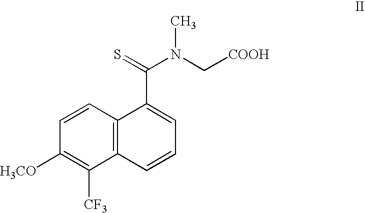

cyclic imides (or more particularly, a hydantoin), such as imirestat (structure III) and sorbinil (structure IV); and

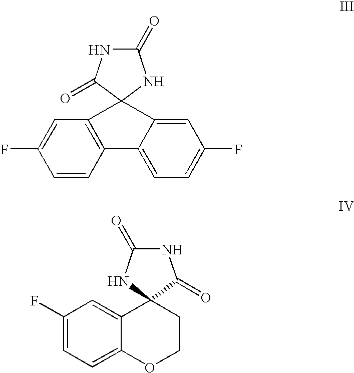

phenylsulfonylnitromethtane derivatives, such as ZD-5522 (structure V).

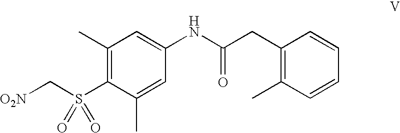

While periodontitis and periodontal disease is a common problem for humans, it may be a worse problem for canines. According to some estimates, eighty percent of dogs are afflicted with some form of periodontal disease. The problem often begins with a build up of plaque on the teeth, progresses to gingivitis, which, left untreated, may progress to periodontitis. If the bone loss becomes severe enough, the dog may be at risk for a jaw fracture, or for a severe systemic bacterial infection. As with humans, the diabetic dog is more at risk for developing periodontal disease than the non-diabetic dog.

Humans seek to avoid severe complications from dental disease by frequent trips to the dentist, brushing the teeth with toothpaste, using mouthwash, and other oral hygiene measures. Dogs, however, depend upon their owners. Many dog owners are unwilling to pay veterinarians for professional teeth cleaning, which is often expensive. Although toothbrushes and toothpaste or dentifrices are available for canines, a great many dog owners are unable to sufficiently control their pets or lack the patience to brush their dog's teeth. Some treats, chews, or dog foods are commercially available that may be of some benefit in keeping a dog's teeth clean. However, such measures are not consistently effective.

Studies have shown that aldose reductase inhibitors may be effective in the prevention and treatment of diabetic cataracts, diabetic neuropathy, and other complications of diabetes. However, none have shown or suggested that aldose reductase inhibitors may be effective in the treatment or prevention of periodontitis. Some dog treats or chews promoted for keeping the dog's teeth clean do contain some quercetin. However, in such applications quercetin is only an adjunct to some other active ingredient, such as Co-enzyme Q, and is present for its antiinflammatory, antioxidant, or free radical scavenging properties, but is not present in sufficient dosage to produce an ARI effect.

Thus, a periodontitis treatment solving the aforementioned problems is desired.

### SUMMARY OF THE INVENTION

The periodontitis treatment is a method for the prevention and treatment of periodontitis in mammals, including humans and dogs. The mammal may be diabetic or non-diabetic. the method includes the step of administering an effective amount of an aldose reductase inhibitor (ARI). The ARI may be (i) a phenolic derivative, such as quercetin, quercitrin (a 3-oxy-glucose analog of quercetin), rutin, and other polyphenols or bioflavonoids exhibiting an ARI effect; (ii) an acetic acid derivative, such as tolrestat, ponalrestat, etc.; (iii) a cyclic imide (or hydantoin), such as sorbinil, 2-methyl sorbinil, imirestat, etc.; and (iv) one of the phenylsulfonylnitromethtane derivatives, such as ZD-5522. In particular embodiments, the method may include, e.g., administering a diet containing about 0.05% quercetin (about 50 mg/kg/day), about 0.0125% imirestat (about 12 mg/kg/day), or about 0.015% tolrestat (about 20 mg/kg/day).

These and other features of the present invention will become readily apparent upon further review of the following specification and drawings.

### BRIEF DESCRIPTION OF THE DRAWINGS

FIG. 1A is a chart of average body weight per week of the study for untreated non-diabetic control rats.

FIG. 1B is a chart of average body weight per week of the study for diabetic rats treated with tolrestat.

FIG. 1C is a chart of average body weight per week of the study for diabetic rats treated with imirestat.

FIG. 1D is a chart of average body weight per week of the study for diabetic rats treated with quercetin.

FIG. 2A is a chart of average dose of tolrestat per week of the study for diabetic rats.

FIG. 2B is a chart of average dose of imirestat per week of the study for diabetic rats.

FIG. 2C is a chart of average dose of quercetin per week of the study for diabetic rats.

FIG. 3 is a chart showing average glycosated hemoglobin levels in each group at the end of the study.

FIG. 4 is a chart showing root/enamel ratio for all groups at the end of the study.

FIG. 5A is a chart of average body weight per week of the study for untreated non-diabetic control rats.

FIG. 5B is a chart of average body weight per week of the study for non-diabetic rats treated with tolrestat.

FIG. 5C is a chart of average body weight per week of the study for non-diabetic rats treated with imirestat.

FIG. 5D is a chart of average body weight per week of the study for non-diabetic rats treated with quercetin.

FIG. 6A is a chart of average dose of tolrestat per week of the study for diabetic rats.

FIG. 6B is a chart of average dose of imirestat per week of the study for diabetic rats.

FIG. 6C is a chart of average dose of quercetin per week of the study for diabetic rats.

FIG. 7 is a chart showing root/enamel ratio for all groups at the end of the study.

Similar reference characters denote corresponding features consistently throughout the attached drawings.

### DETAILED DESCRIPTION OF THE PREFERRED EMBODIMENTS

The periodontitis treatment is a method for the prevention and treatment of periodontitis in mammals, including humans and dogs. The mammal may be diabetic or non-diabetic. the method includes the step of administering an effective amount of an aldose reductase inhibitor (ARI). The ARI may be (i) a phenolic derivative, such as quercetin, quercitrin (a 3-oxy-glucose analog of quercetin), rutin, and other polyphenols or bioflavonoids exhibiting an ARI effect (a number of such compounds are reviewed by de la Fuente and Manzanaro in “Aldose reductase inhibitors from natural sources”, Nat. Prod. Rep., 20, 243-251 (2003)); (ii) an acetic acid derivative, such as tolrestat, ponalrestat, etc.; (iii) a cyclic imide (or hydantoin), such as sorbinil, 2-methyl sorbinil, imirestat, etc.; and (iv) one of the phenylsulfonylnitromethtane derivatives, such as ZD-5522. In particular embodiments, the method may include, e.g., administering a diet containing about 0.05% quercetin by weight (about 50 mg/kg/day), about 0.0125% imirestat by weight (about 12 mg/kg/day), or about 0.015% tolrestat by weight (about 20 mg/kg/day).

It is noted that the commercial version of tolrestat (Alredase) was withdrawn from the market by the manufacturer (Wyeth) in 1996 or 1997, apparently in the wake of reports of fatalities from hepatic necrosis and the failure of later clinical trials to confirm the results of earlier trials. However, the dosage levels herein are believed to be within safe limits.

Current dental treatment for diabetic periodontal disease is primarily focused on the control of oral infections. Overlooked have been the potential contribution of the enzyme aldose reductase (AR) and the sorbitol pathway, which plays a critical role in the development of diabetic complications associated with neuropathy, nephropathy and micro and macroangiopathy. Several studies have shown that ARIs are effective in the prevention and treatment of several of the general complications of diabetes discussed above. In the course of investigating the role of ARIs in the prevention and treatment of various complications of diabetes, the present inventor noticed and demonstrated that imirestat is effective in reducing alveolar bone loss associated with periodontitis in diabetic rats. The following studies summarized in Examples 1 and 2 resulted.

### EXAMPLE 1

A group of 80 approximately 81-100 g male Sprague Dawley rats were utilized for this study. Diabetes was induced in 40 of these rats by tail vein injection of 75 mg/kg of streptozotocin. After 3 days, blood glucose (BG) levels in blood obtained from tail vein lacerations were analyzed with a commercial glucometer (Freestyle by TheraSense, Alameda, Calif.). Each rat with BG levels <300 mg/dL were reinjected with streptozotocin. Again, BGs were measured after 3 days, and the procedure was repeated a third time for remaining rats with BGs <300 mg/dL.

All rats with blood glucose levels >300 mg/dL were then equally divided into 4 groups. The first diabetic group of 8 rats received standard rat diet (BioServe), the second received similar rat diet containing 0.015% of tolrestat, the third group received similar diet containing 0.0125% imirestat, and the fourth group received similar diet containing 0.08% quercetin. Experimental diets were initiated 10 days following initial streptozotocin injections and continued for 10 weeks until the studies were terminated. Non-diabetic age-matched rats were also added as additional non-diabetic controls. These consisted of 4 groups of 8 rats each as follows: an untreated non-diabetic group received standard rat diet, the second control group received similar rat diet containing 0.015% of tolrestat, the third control group received similar diet containing 0.0125% imirestat, and the fourth control group received similar diet containing 0.08% quercetin.

A maximum of 3 rats were housed per cage. Weekly consumption of diet/cage and body weights of each cage occupant was measured throughout the study. Drugs diets were prepared by adding the appropriate weight of drug to 3 kg portions of standard rat chow as follows. The weighed drug dissolved in 400 mL of ethanol was uniformly sprayed onto a monolayer of the rat chow. After the upper layer of chow was uniformly wetted, the chow was mixed and again formed into a monolayer, and the procedure was repeated until the entire drug solution was utilized. Following overnight oven drying to ensure that all solvent was removed, weighed amounts of diet were administered to each appropriate group of animals.

The procedure for drug diet preparation using either tris buffer or alcohol was initially established in the 1970's by Dr. Jin Kinoshita at the National Eye Institute, National Institutes of Health, Bethesda Md. This dosing has the advantage of minimizing potential drug half-life effects, since rats continuously feed while active, and thus receive continuous doses of drug. Moreover, since the drug diet is prepared weekly, any decreases of drug potency due to limited shelf-life are minimized.

Forty-four days after initiation of diets, each rat was anesthetized with 4% isoflurane vapor in an induction chamber. Each rat was then placed on its back and the palatal gingiva between the first and second maxillary molars on the right side was injected with 10 μL of a phosphate-buffered saline (PBS) solution containing 1 mg/mL lipopolysaccharide LPS from Porphyromonas gingivalis (InvivoGen, San Diego, Calif.) using an insulin syringe equipped with a blunted edge 30-gauge needle to induce the formation of periodontitis. As a control, 10 μL of PBS was similarly injected into the left side of the jaw. This injection was followed by two additional injections at 48-hour intervals. Twenty-four days after the final injection, all rats were euthanized with carbon dioxide gas. The heads were decapitated with a guillotine and frozen for subsequent defleshing. Blood from each rat was obtained by heart puncture and HbA1c levels were measured using the Metrika A1cNOW Plus System Test Strips (Fisher Healthcare).

Each thawed head was manually defleshed using standard dental instruments by Dr. James O'Meara, Assoc. Professor of General Dentistry at Creighton University of Omaha, Nebr. The maxillary area was separated from the remaining skull using bone cutters, and each defleshed left and right maxillary alveolus with molars were stored in PBS buffer. Finally, all cleaned maxillary alveoli were placed in 3% aqueous peroxide solution overnight and air dried. They were then stained in 5% aqueous toluidine blue to identify the cemento-enamel junction (CEJ) on the molars. After air drying, each stained preparation was examined under a dissecting microscope and cleaned of any remaining flesh particles using the tip of a 20-gauge needle. Each palatal preparation was then placed onto a 20 mm petri dish containing ultrafine chromatography grade silica and a mm ruler. The maxilla-molar area palatal sides were then photographed using a PAXcam2+ USB2 Digital Microscope Camera attached to a Zeiss dissection microscope at 40' magnification. 2.0 MP images were captured with 1616×1216 image resolution. The images were analyzed using Pax-it software (Paxcam, Chicago, Ill.). Care was taken to obtain images with consistent camera angles.

In each image the two molars between which the injections were made were analyzed. The exposed side of each molar was manually outlined along with the area encompassed by the cemento-enamel junction (CEJ) and exposed root area. To facilitate differentiation of the exposed root area and the CEJ, each photograph was photo-enhanced as required using sharpening or desaturating techniques. Multiple tracings were obtained and averaged for each image. Moreover, since tooth movement can also increase variability, multiple images of the same sample were also analyzed, if necessary. The area measurements from both molars were combined, and from these combined area measurements, a ratio of exposed root area (root to the CEJ) versus enamel area was obtained. Ratios for both the right (LPS) and left (PBS) injected sides from the same rat were compared to confirm the integrity of the results, since bone loss in the LPS side could not be less than that of the PBS side, which is the internal control. The results were statistically evaluated using SigmaPlot version 11 software (Systat Software, Inc, San Jose Calif.).

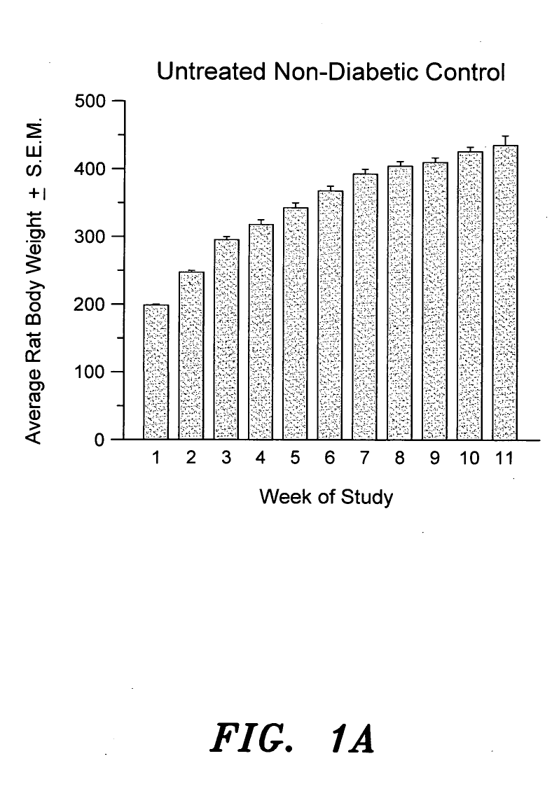

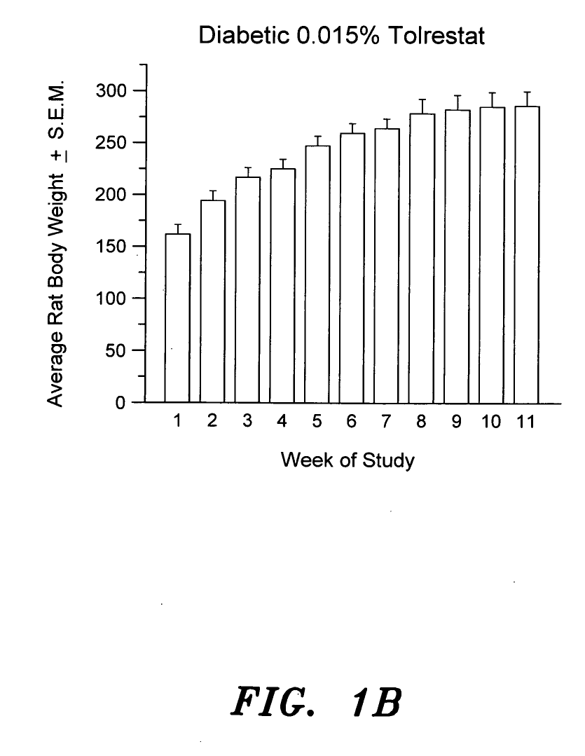

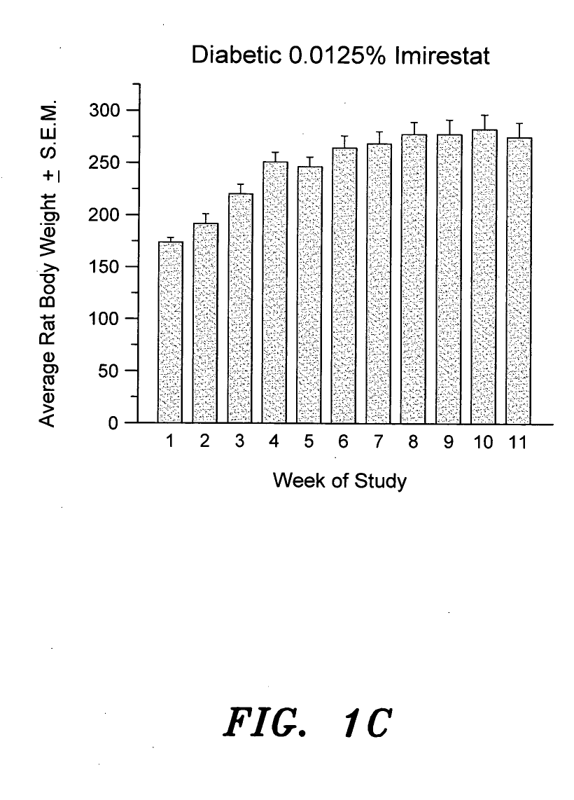

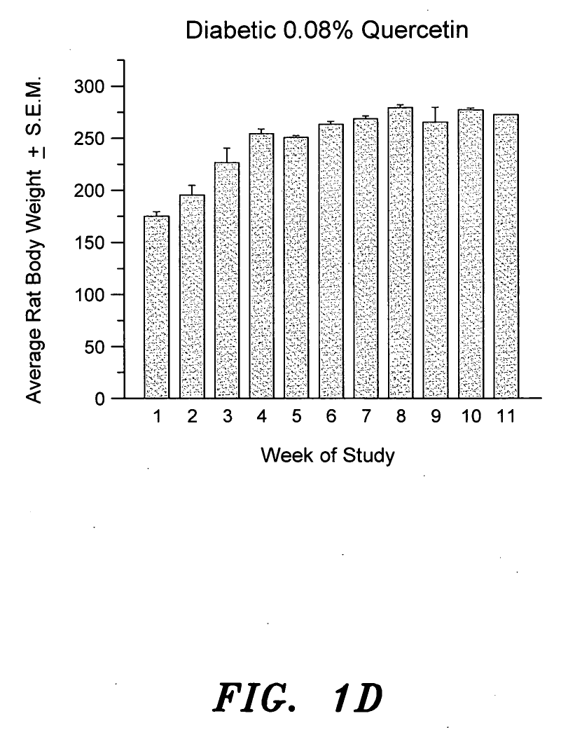

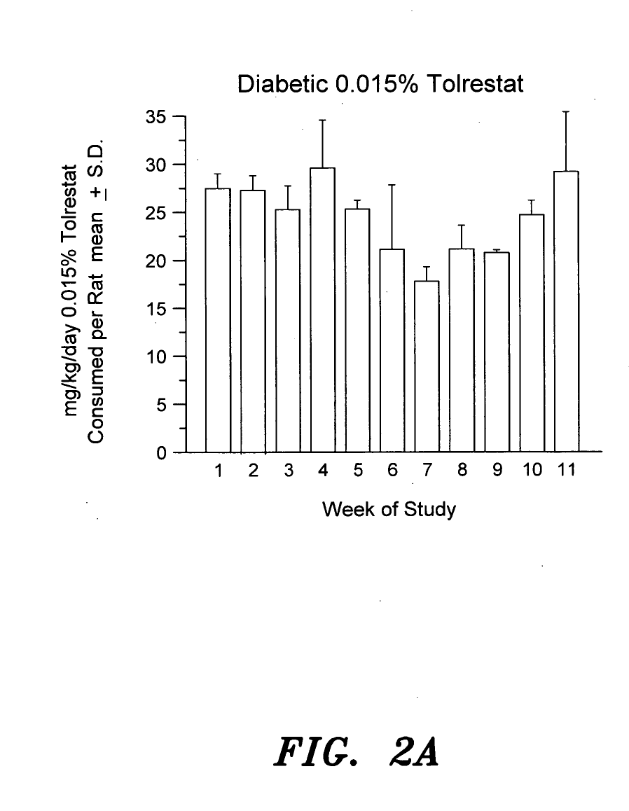

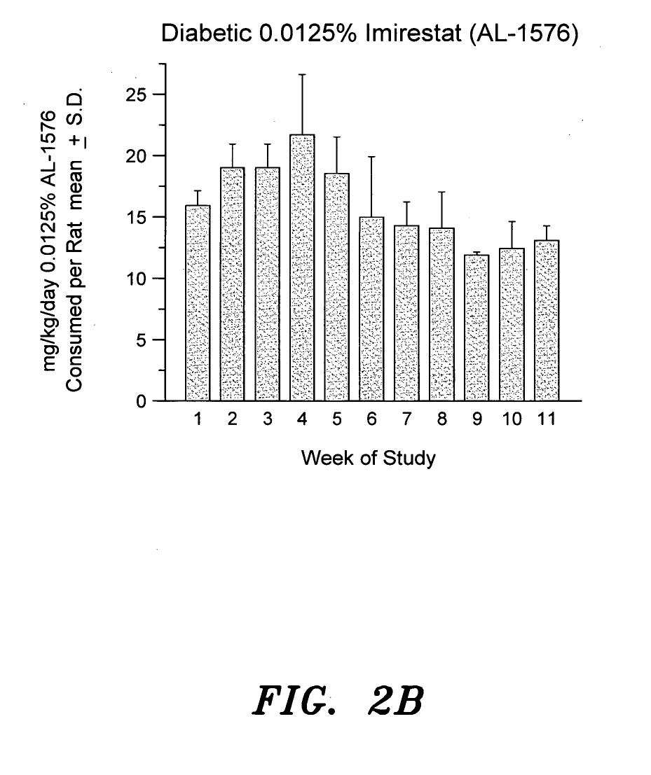

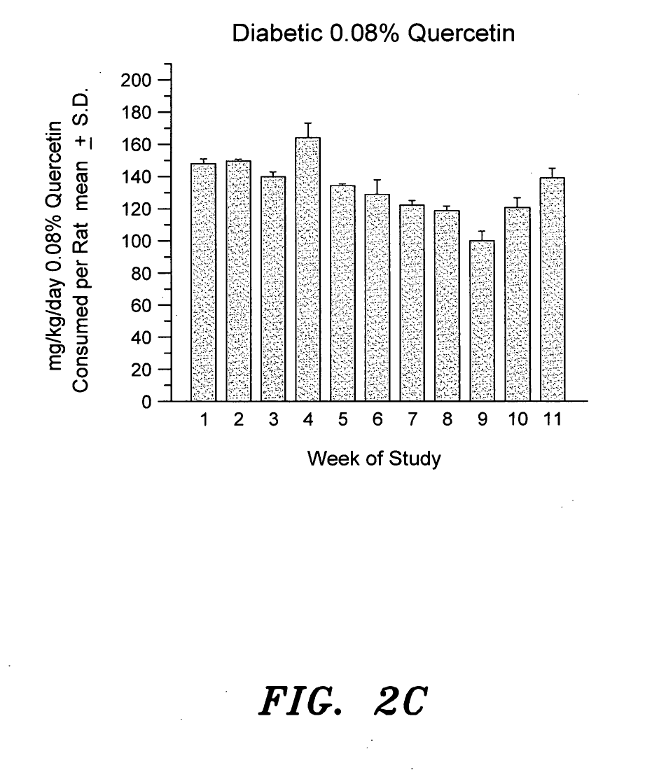

Tail vein injection of streptozotocin (75 mg/kg) to the young Sprague Dawley rats resulted in the induction of diabetes, with all rats demonstrating blood glucose levels >300 mg/dL. These rats were randomly divided into 4 groups containing a minimum of 8 rats/group. Experimental diets were initiated 10 days after the initial induction of diabetes. The 4 groups were dosed as follows. The first untreated diabetic group received standard rat diet, the second received standard rat diet containing 0.015% of tolrestat, the third group received standard diet containing 0.0125% imirestat, and the fourth group received standard diet containing 0.08% quercetin. Additional groups of 8 age-matched non-diabetic control rats received a standard control diet with/without aldose reductase inhibitors. Average drug doses received were estimated from weekly consumption of diet/cage and body weights of each cage occupant. As summarized in FIGS. 1A-1D, weight gains were similar in all diabetic groups. Based on weekly diet consumption and body weights, the average dose of drug weekly ingested is summarized in FIGS. 2A-2C. From these studies the average estimated dose of aldose reductase inhibitors ingested by each rat was tolrestat 23.9±4.56; imirestat 16.4±0.9 and quercetin 133.±5.6 mg/kg/day (mean±S.D.).

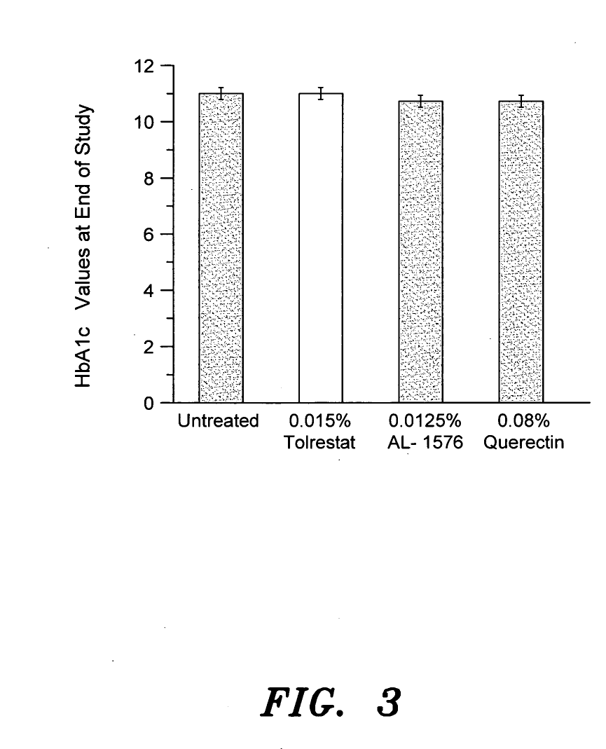

After 44-days of diet, each rat was anesthetized and injected 3 times at 48-hour intervals on the right inside palatial gingiva between the first and second maxillary molar with 10 μL of a PBS solution containing 1 mg/mL lipopolysaccharide from Porphyromonas gingivalis. As a control, the left side was similarly injected with 10 μL of a PBS solution. The significantly more expensive lipopolysaccharide from human Porphyromonas gingivalis rather than the previously utilized lipopolysaccharide from Escherichia coli 055:B5 9 (Aldrich, St. Louis, Mo.) was used at the suggestion of experts on the NIH Dental Institute Study Section because the periodontitis induced with Porphyromonas gingivalis is believed to be more similar to the severity of human periodontitis. Twenty-four days after the final injection, all rats were euthanized with carbon dioxide gas and decapitated. Analysis of HbA1c levels, illustrated in FIG. 3, verified that all rats were equally diabetic for the duration of the study with HbA1c levels as follows: untreated diabetic 10.95±36; diabetic tolrestat 10.76±0.45; diabetic imirestat 10.48±0.86; diabetic quercetin 10.45±0.28 (mean±S.E.M.)

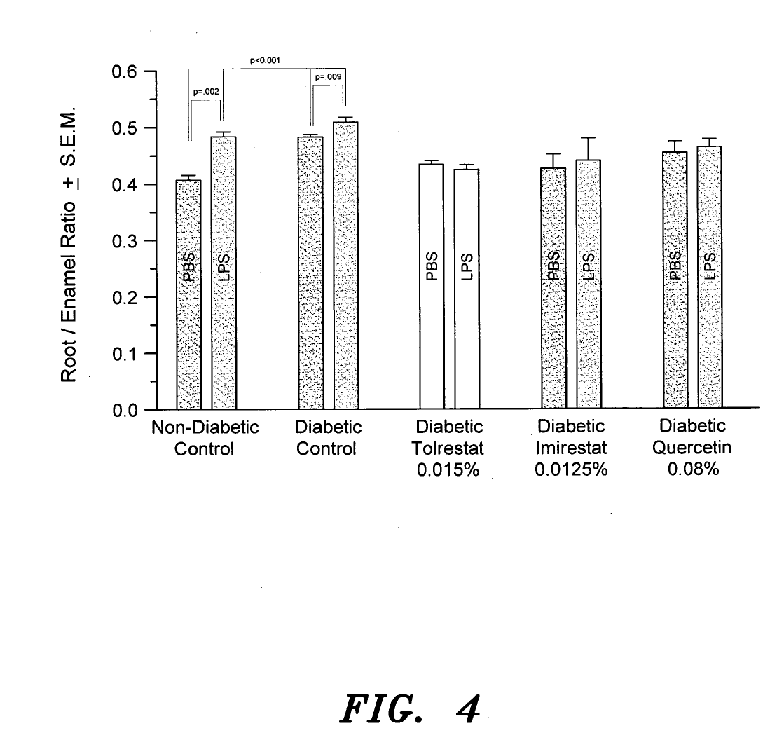

Analyses were conducted by manually tracing the total inside area of encompassed by the root and to CEJ area of the molars in each stained palatal preparation. These area values were then used to calculate the ratio of exposed root to enamel area of the combined molars between injections. This ratio is representative of alveolar bone loss since increased exposed root area results in an increased ratio. The ratios obtained for the PBS and LPS sides of each group are (mean±S.E.M): non-diabetic control 0.421±0.010 and 0.477±0.014; non-treated diabetic control 0.475±0.006 and 0.511±0.012; tolrestat 0.435±0.010 and 4.22±0.011; imirestat 0.421±0.029 and 0.439±0.043 and quercetin 0.440±0.023 and 0.448±0.057. As summarized in FIG. 4, the results clearly show that LPS injections result in higher root/enamel ratios versus PBS injections in both the non-diabetic and diabetic untreated control rats. Comparisons of LPS to PBS injected sides were conducted using t-test analyses following a Shapiro-Wilk normality test for each set of data. Statistical differences between the LPS versus PBS sides were obtained for the non-diabetic controls (p=0.002) and untreated diabetic groups (p=0.009). Statistical comparisons of all rat groups using multiple ANOVA analysis with the untreated PBS injected side of non-diabetic control rats designated as the control indicated that only the LPS injected sides of the non-diabetic rats, as well as both the LPS and PBS sides of the diabetic rats, were significantly different (p<0.001). These results indicate that treatment with all aldose reductase inhibitors examined equally prevented alveolar bone loss.

### EXAMPLE 2

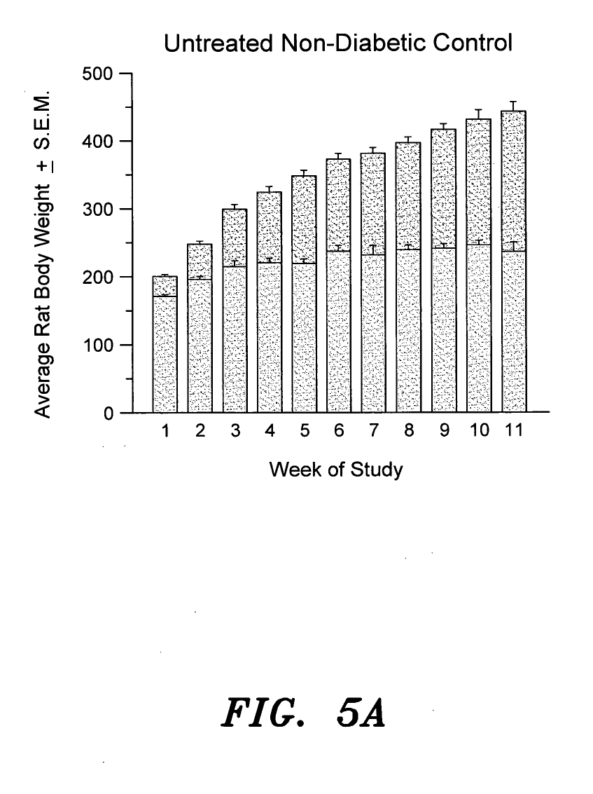

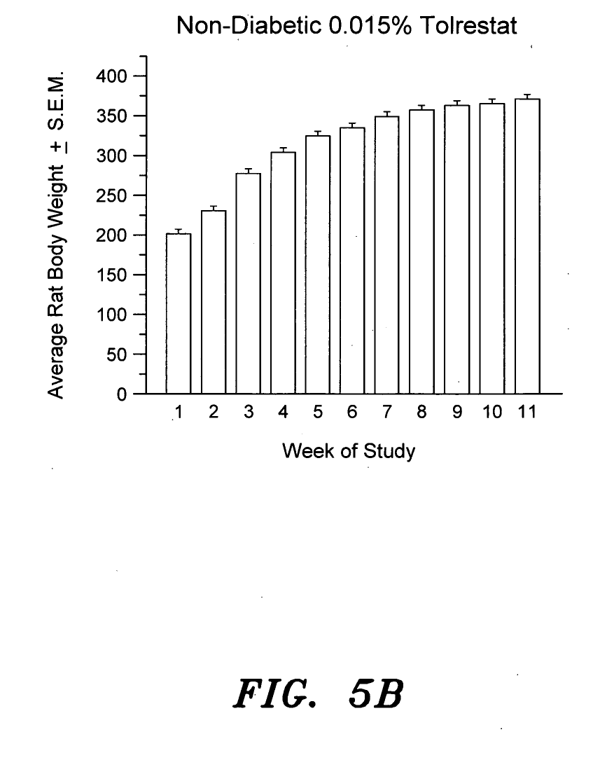

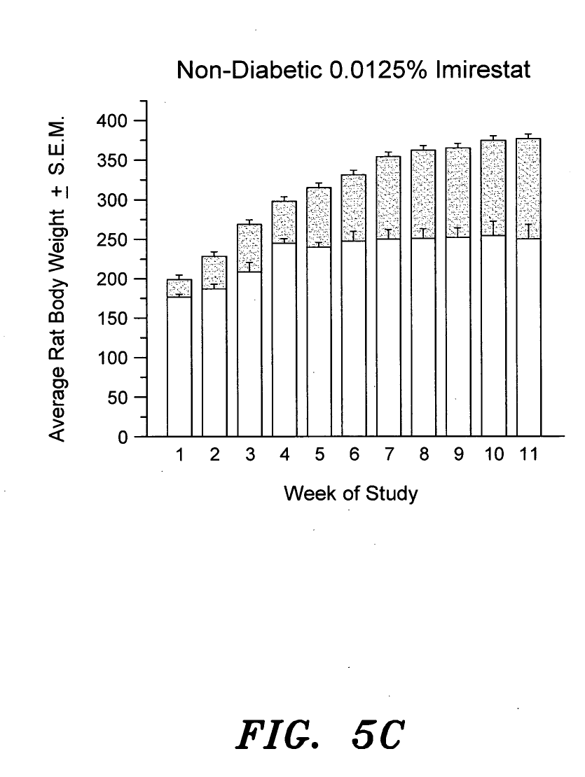

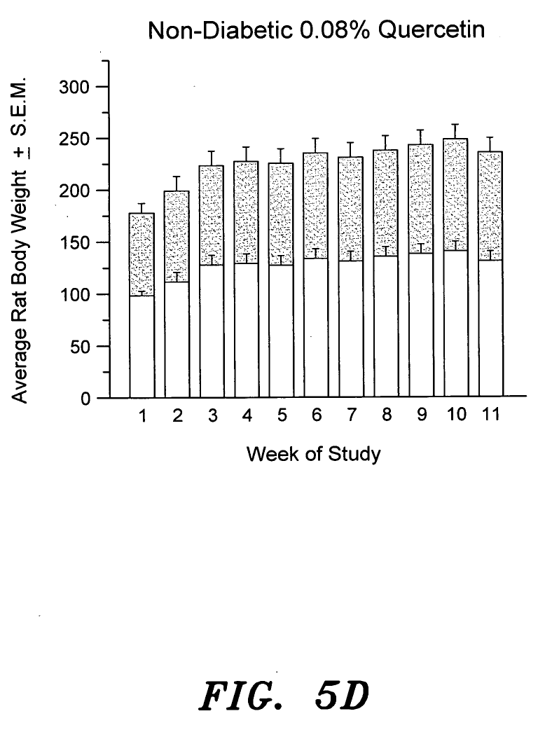

The study described in Example 1 was extended to investigate the effects of ARIs on non-diabetic rats using substantially the same protocol. In the previous study, 4 groups of non-diabetic rats containing a minimum of 8 rats/group were used as additional controls to observe the effects of aldose reductase inhibitors on loss of alveolar bone in non-diabetic rats. The 4 groups were dosed as follows. The first untreated non-diabetic group received standard rat diet, the second received standard rat diet containing 0.015% of tolrestat, the third group received standard diet containing 0.0125% imirestat, and the fourth group received standard diet containing 0.08% quercetin. Average drug doses received were estimated from weekly consumption of diet/cage and body weights of each cage occupant. As summarized in FIGS. 5A-5D, weight gains appeared similar in all non-diabetic groups. However, weight gain was significantly greater that in the diabetic group.

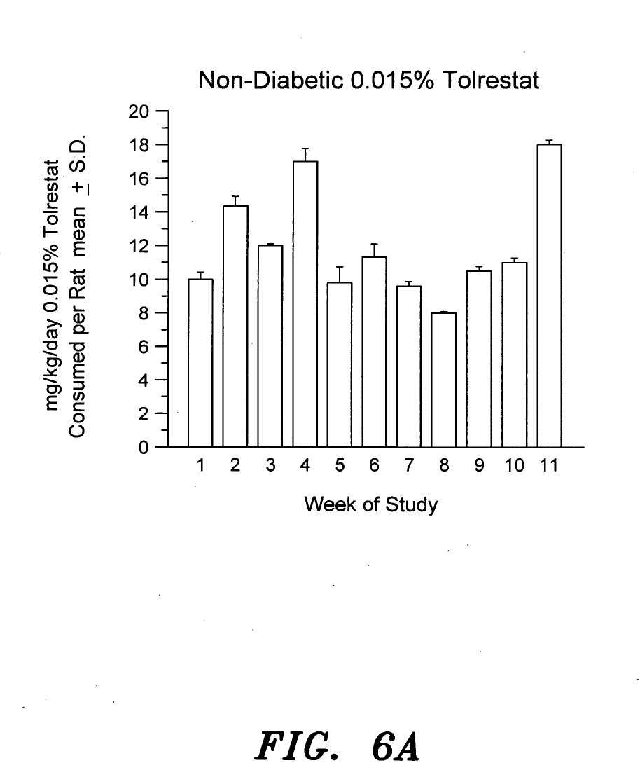

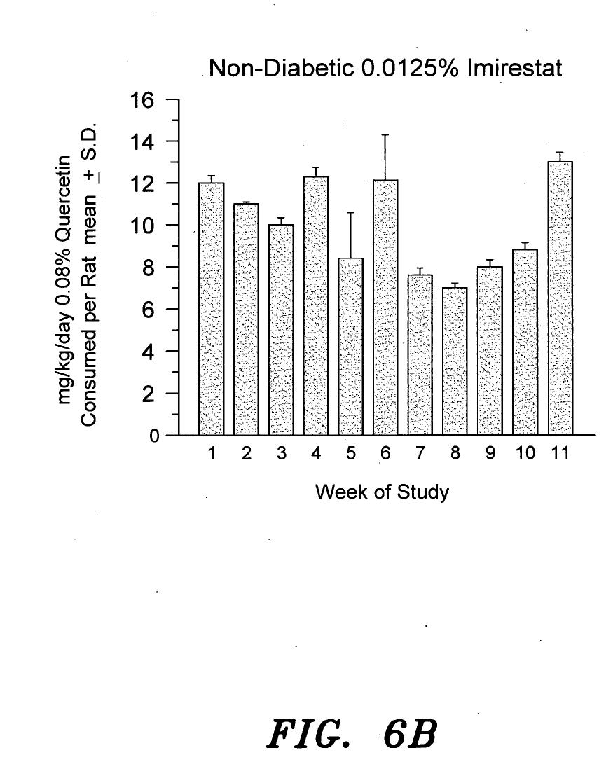

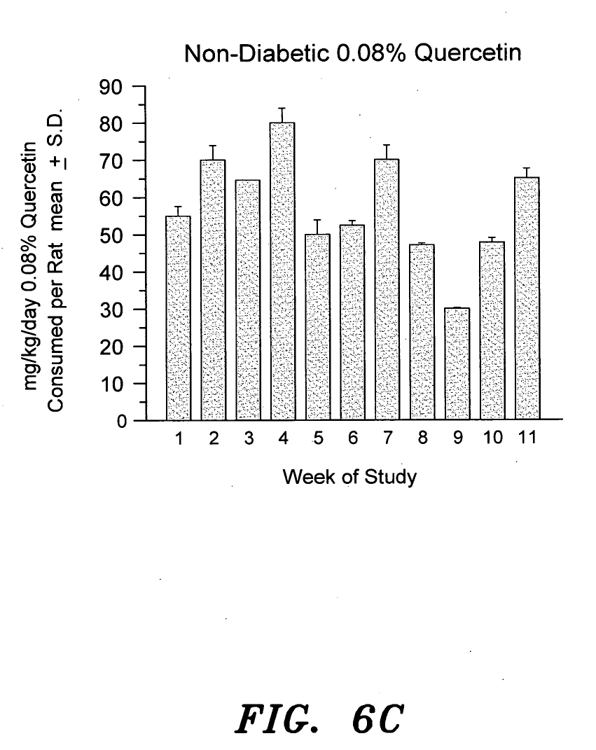

Based on weekly diet consumption and body weights, the average dose of drug weekly ingested by the non-diabetic rats is summarized in FIGS. 6A-6C. Over the 11-week period, the average calculated dose of tolrestat ingested was 12.0±0.94 mg/kg/day, imirestat 10.8±2.4 mg/kg/day and the lower dose of quercetin 58.0±4 mg/kg/day. Although each rat had access to unlimited amounts of diet, both the diabetic and non-diabetic rats ingested similar amounts of diet. Therefore, the unexpectedly lower doses of drug ingested by the non-diabetic rats are due to their higher body weights.

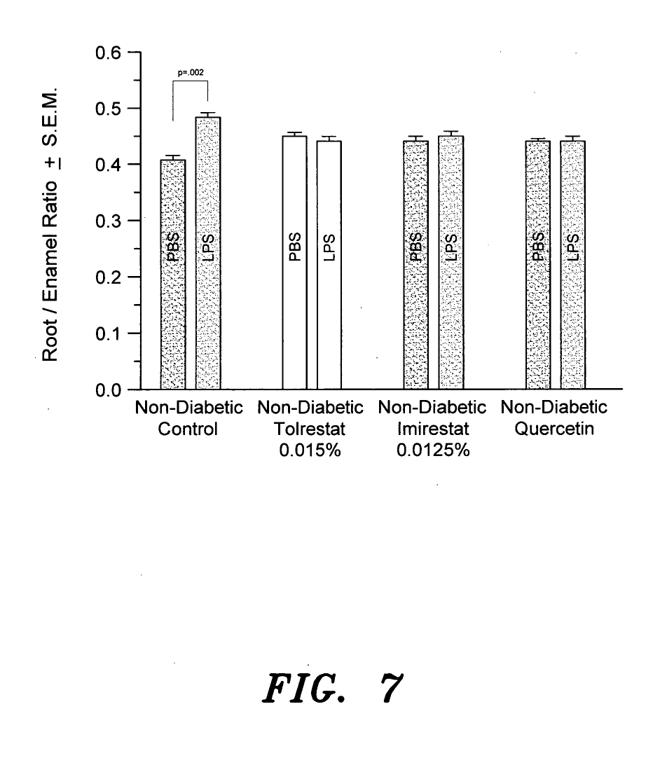

Analyses were conducted by manually tracing the total inside area of encompassed by the root and to CEJ area of the molars in each stained palatal preparation. From these areas, the ratio of exposed root to enamel area of the combined molars between injections was calculated. In all normal rats treated with either tolrestat, imirestat or quercetin, no significant difference in the ratios between the LPS and PBS sides were observed, as shown in FIG. 7. The respective ratios were: tolrestat 0.435±0.039 and 0.422±0.036; imirestat 0.412±0.016 and 0.439±0.0132; quercetin 0.406±0.018 and 0.417±0.042.

Based on our present results, the efficacy of these structurally diverse aldose reductase inhibitors in periodontal disease are based on their ability to inhibit aldose reductase and as a result of this inhibition, they are also able to mediate inflammatory responses that are downfield of aldose reductase. The results in Example 2, as shown in Example 2, demonstrate that aldose reductase inhibitors are equally effective in preventing alveolar bone loss in non-diabetic subjects.

Although the experimental results in Examples 1 and 2 are limited to three particular compounds, the facts that these compounds are so structurally dissimilar, not members of a common genus of chemical compounds, and apparently have nothing in common but the common property of exhibiting the effect of inhibiting aldose reductase activity lead to the prediction that any aldose reductase inhibitor, regardless of structure, should be effective in the prevention and treatment of periodontitis in mammals. The exact dosage ranges required to achieve the desired effect for a given ARI may be determined by routine experimentation according to the methods illustrated by Examples 1 and 2.

For the treatment of canines, the aldose reductase inhibitor may be incorporated into a food product, such as dog food, dog treats, dog chews, or the like.

It is to be understood that the present invention is not limited to the embodiments described above, but encompasses any and all embodiments within the scope of the following claims.

I claim:

1. A method for prevention and treatment of periodontitis in a mammal, comprising the step of administering an aldose reductase inhibitor to the mammal in an amount effective to reduce alveolar bone loss normally resulting from periodontitis.

2. The method for prevention and treatment of periodontitis according to claim 1, wherein the mammal is a human.

3. The method for prevention and treatment of periodontitis according to claim 1, wherein the mammal is a canine.

4. The method for prevention and treatment of periodontitis according to claim 1, wherein the mammal is diabetic.

5. The method for prevention and treatment of periodontitis according to claim 1, wherein the aldose reductase inhibitor is a phenolic derivative.

6. The method for prevention and treatment of periodontitis according to claim 1, wherein the aldose reductase inhibitor is a polyphenol.

7. The method for prevention and treatment of periodontitis according to claim 1, wherein the aldose reductase inhibitor is a bioflavonoid.

8. The method for prevention and treatment of periodontitis according to claim 1, wherein the aldose reductase inhibitor comprises quercetin.

9. The method for prevention and treatment of periodontitis according to claim 1, wherein the aldose reductase inhibitor comprises quercetin, the step of administering further comprising the step of including about 0.08% quercetin by weight in the diet of the mammal.

10. The method for prevention and treatment of periodontitis according to claim 1, wherein the step of administering further comprises the step of administering a diet containing quercetin in an amount of about 50 mg/kg/day.

11. The method for prevention and treatment of periodontitis according to claim 1, wherein the aldose reductase inhibitor is a cyclic imide.

12. The method for prevention and treatment of periodontitis according to claim 1, wherein the aldose reductase inhibitor is a hydantoin derivative.

13. The method for prevention and treatment of periodontitis according to claim 1, wherein the aldose reductase inhibitor comprises imirestat.

14. The method for prevention and treatment of periodontitis according to claim 1, wherein the aldose reductase inhibitor comprises imirestat, the step of administering further comprising the step of including about 0.0125% imirestat by weight in the diet of the mammal.

15. The method for prevention and treatment of periodontitis according to claim 1, wherein the step of administering further comprises the step of administering a diet containing imirestat in an amount of about 12 mg/kg/day.

16. The method for prevention and treatment of periodontitis according to claim 1, wherein the mammal is a canine, the method further comprising the step of incorporating the aldose reductase inhibitor in a food product selected from the group consisting of dog food, dog treats, and dog chews, the step of administering further comprising the step of including the food product in the diet of the canine.

17. A method for prevention and treatment of periodontitis in a mammal, comprising the step of administering quercetin to the mammal in an amount effective to inhibit aldose reductase activity in order to reduce alveolar bone loss normally resulting from periodontitis.

18. The method for prevention and treatment of periodontitis according to claim 17, wherein the step of administering further comprises the step of administering a diet containing quercetin in an amount of about 50 mg/kg/day.

19. A method for prevention and treatment of periodontitis in a mammal, comprising the step of administering imirestat to the mammal in an amount effective to inhibit aldose reductase activity in order to reduce alveolar-bone loss normally resulting from periodontitis.

20. The method for prevention and treatment of periodontitis according to claim 19, wherein the step of administering further comprises the step of administering a diet containing imirestat in an amount of about 12 mg/kg/day.
